(19) United States
(12) Patent Application Publication
(10) Pub. No.: US 2025/0090144 A1
(43) Pub. Date: Mar. 20, 2025

(54) SYSTEM AND METHOD FOR CHARACTERIZING ULTRASOUND DATA

(71) Applicant: ONCOUSTICS INC., Toronto (CA)

(72) Inventors: Adi LIGHTSTONE, Toronto (CA); Ahmed EL KAFFAS, Toronto (CA); Beth ROGOZINSKI, Toronto (CA); Miriam NAIM IBRAHIM, Toronto (CA); Raul BLÁZQUEZ GARCÍA, Toronto (CA)

(21) Appl. No.: 18/721,115

(22) PCT Filed: Dec. 19, 2022
(86) PCT No.: PCT/CA2022/051856

§ 371 Date: Jun. 17, 2024

Related U.S. Application Data
(60) Provisional application No. 63/290,963, filed on Dec. 17, 2021.

(51) Int. Cl. A61B 8/08; A61B 8/00; G06T 7/00; G16H 30/40

(52) U.S. Cl. CPC A61B 8/5223; A61B 8/0825; A61B 8/46; A61B 8/485; A61B 8/5292; G06T 7/0012; G16H 30/40; G06T 2207/10132; G06T 2207/20081; G06T 2207/30056; G06T 2207/30068; G06T 2207/30081; G06T 2207/30084; G06T 2207/30088; G06T 2207/30092; G06T 2207/30096

(57) ABSTRACT

A system and method for characterising tissues are provided. The system comprises a point-of-care ultrasound device for obtaining ultrasound images of tissues within a system of interest, a processor, and a memory comprising instructions which when executed by the processor configure the processor to perform the method. The method comprises obtaining an ultrasound image of a tissue types within a system of interest; identifying features of the tissue on the ultrasound image, feeding said identified features to a trained model, and identifying a tissue pathology based on the identified features.

### CROSS-REFERENCE TO RELATED APPLICATIONS

This claims the benefit of U.S. Provisional Patent Application No. 63/290,963, filed on Dec. 17, 2021, the entire contents of which are incorporated by reference herein.

### FIELD

The present disclosure generally relates to artificial intelligence, and in particular to a system and method for characterizing biological tissues with ultrasound data.

### INTRODUCTION

Hepatic (liver) fibrogenesis is a common response to chronic liver diseases (CLDs) that can lead to advanced fibrosis (cirrhosis) and ultimately to liver failure. Etiologies that result in hepatic injury, starting with inflammation and leading to progressive fibrosis, are expected to drive most liver-related cancers, liver transplants and/or mortalities from end stage liver disease in the next decade. Recent studies have shown the prevalence of liver fibrosis in the general adult population without previously known liver diseases is high (ranging up to 9%), and the prevalence among individuals with risk factors is much higher (up to 27.9%), mostly being driven by the obesity epidemic leading to non-alcoholic fatty liver disease (NAFLD). Without early detection and intervention, liver fibrosis advances silently and can lead to potentially irreversible conditions like cirrhosis, portal hypertension and liver failure that result in increased disability, liver cancer, liver transplants, and early patient death.

If diagnosed early, the underlying cause of liver fibrosis can be treated, allowing patients to outlive their liver disease. Several studies have demonstrated that liver fibrosis can even be reversed with existing and emerging treatment options. Yet, as liver fibrosis itself is essentially asymptomatic until very advanced stages, early diagnosis can be delayed due to lack of symptoms and the unavailability of non-invasive diagnostic tools. Given this situation, apart from patients with known liver disease or viral infections, such as hepatitis B virus (HBV) or hepatitis C virus (HCV), fibrosis is not screened for despite the clinical implications of the condition.

None of the available modalities that can detect liver fibrosis are suitable or effective for early disease screening. Blood tests, including traditional liver function panels, have been shown to miss even significant fibrosis and cirrhosis in the majority of patients. A suite of new serum biomarker tests have likewise been shown to have significant limitations in the assessment of liver fibrosis. Transient elastography, used to perform fibrosis surveillance on patients with HBV/HCV infection(s) offers the potential to diagnose and stage liver fibrosis in subspecialty hepatology clinics, but such tools are not available in primary care or other settings suitable for screening; therefore, by the time patients reach a specialty level of care where transient elastography is applied, they are usually at a much more advanced stage of disease.

To enable screening for pre-symptomatic fibrosis, there is an unmet need for a tool that can be used broadly in high-risk patient groups, outside of hepatology subspecialty clinics, so that patients can be identified early when the condition can be treated. Available tools are either not sufficiently accurate for early stage disease or cannot be applied broadly enough to be suitable for screening. A tool that addressed these limitations to enable early identification of disease could ultimately decrease morbidity and help save lives.

### SUMMARY

A system is provided that includes a point of care ultrasound that can yield raw ultrasound data (i.e. beamformed or channel radio-frequency ultrasound backscattered data), a data acquisition protocol designed to maximize on tissue views (e.g., liver) through a series of sweeps throughout the abdomen that captures hundreds to thousands of images, and an artificial intelligence (AI) algorithm trained to recognize featured related to tissues and diseases/pathology from the raw ultrasound data. Disease that causes tissue morphological or structural changes may be detected either directly or via correlations to physical measures such as estimates of tissue stiffness (as represented in kiloPascals (kPa), as well as measurements of the ultrasound coefficient of attenuation. The system can be built by obtaining a large data set of raw ultrasound data acquired through the data acquisition protocol using a point of care ultrasound system. The data is then used to train a machine learning model that identifies good frames from bad frames, a machine learning algorithm that provides estimates of tissue stiffness as well as measurements of the ultrasound coefficient of attenuation, and/or identifies tissues within all images acquired through the acquisition protocol, and/or a machine learning algorithm that identifies specific tissue pathologies or disease of interest in the tissue. Training is done on data with appropriate labels.

In accordance with an aspect, there is provided a system for providing estimates of tissue stiffness as well as measurements of the ultrasound coefficient of attenuation and/or characterising tissues. The system comprises a point-of-care ultrasound device for obtaining ultrasound images of a tissue, a processor, and a memory comprising instructions which when executed by the processor configure the processor to obtain an ultrasound image of a tissue, identify features of the tissue on the ultrasound image, feed said identified features to a trained model, and identify a tissue pathology based on the identified features.

In accordance with another aspect, there is provided a method of providing estimates of tissue stiffness, measurements of ultrasound coefficient of attenuation, and/or characterising tissues. In some embodiments, the method comprises obtaining an ultrasound image of a tissue, identifying features of the tissue on the ultrasound image, feeding said identified features to a trained model, and identifying a tissue pathology based on the identified features.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates, in a component diagram, an example of an ultrasound system, in accordance with some embodiments;

FIG. 2 illustrates an example of an input and an output of an AI subsystem, in accordance with some embodiments;

FIG. 3 illustrates, in a schematic diagram, an example of a machine learning platform for characterizing ultrasound data, in accordance with some embodiments;

FIGS. 4 to 6 illustrate an ultrasound image and a corresponding scan angle, in accordance with some embodiments;

FIGS. 7 and 8 illustrate, in screen shots, examples of data acquisition and scan results, in accordance with some embodiments;

FIG. 9 illustrates an example of a liver segmentation model, in accordance with some embodiments;

FIG. 10 illustrates, in a timeline diagram, an example of a processing pipeline, in accordance with some embodiments;

FIGS. 11 to 14 illustrate examples of the initial results page, in accordance with some embodiments;

FIG. 15 illustrates, in a data flow diagram, an example of a method of data collection enrolment, in accordance with some embodiments;

FIGS. 16 and 17 illustrates distributions of Age and BMI in the patient data set used to develop and test the models, in accordance with some embodiments;

FIG. 18 illustrates an example of ML training, in accordance with some embodiments;

FIG. 19 illustrates, in a graph, an example of a model ROC for training data set, in accordance with some embodiments;

FIG. 20 illustrates, in a graph, an example of reserve data set ROC curves, in accordance with some embodiments;

FIG. 21 illustrates, in a graph, an example of survival analysis of patients, in accordance with some environments;

FIG. 22 illustrates an example of a system stage shift vs the standard of care for high risk patients, in accordance with some embodiments;

FIG. 23 illustrates, in process flowcharts, a comparison of a system fibrosis test vs a process of requesting lab work; and

FIG. 24 is a schematic diagram of a computing device such as a server or other computer in a device such as a vehicle.

It is understood that throughout the description and figures, like features are identified by like reference numerals.

### DETAILED DESCRIPTION

Embodiments of methods, systems, and apparatus are described through reference to the drawings. Applicant notes that the described embodiments and examples are illustrative and non-limiting. Practical implementation of the features may incorporate a combination of some or all of the aspects, and features described herein should not be taken as indications of future or existing product plans.

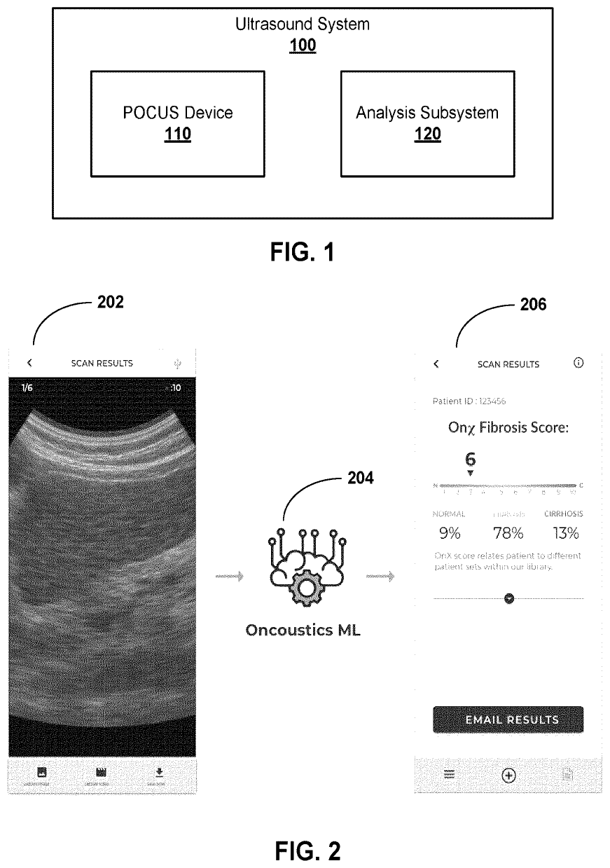

FIG. 1 illustrates, in a component diagram, an example of an ultrasound system 100, in accordance with some embodiments. The system 100 comprises a point-of-care ultrasound (POCUS) device 110 and an analysis subsystem 120. In some embodiments, the analysis subsystem 120 may comprise an artificial intelligence (AI)/machine learning (ML) subsystem. Other components may be added to the system 100.

In some embodiments, a software as a medical device (SaMD) solution is provided that uses ultrasound radio frequency (RF) signal data to measure tissue acoustic properties obtained from a POCUS ultrasound device. In some embodiments, it is set up to work with a handheld ultrasound device connected to a smart phone or other computing device having a display). However, other ultrasound probes or POCUS devices may be used. RF data is typically obtained during ultrasound acquisition, but quickly discarded after conventional ultrasound images are generated. In contrast, the proposed solution stores and uses this RF data to obtain the acoustic properties of the imaged tissue, and to provide computer analytics based on quantitative ultrasound (QUS) and radiomic parameters. These imaging features are then synthesized by an artificial intelligence algorithm into a single value or metric—the “OnX” score. This score relates any subject patient to different normative patient sets within a library, and the proportion of Normal, Fibrotic, and Cirrhotic patients in each score group may be displayed as a percentage, as shown in FIG. 2. In some embodiments, the score can be presented as an estimate of tissue stiffness and presented as kPa as well as measurements of the ultrasound coefficient of attenuation and/or presented as a categorization tool which, for example, displays the proportion of Normal, Fibrotic, and Cirrhotic patients in each score group as a percentage (as depicted in FIG. 2). FIG. 2 illustrates an example of an input 202 and an output 206 of an AI subsystem 204, in accordance with some embodiments. In some embodiments, the AI may be trained on over 2,000,000 data points from over 4,000 individual patients determined to be normal or having fibrotic or cirrhotic tissues. In some embodiments, outputs may include kPa outputs.

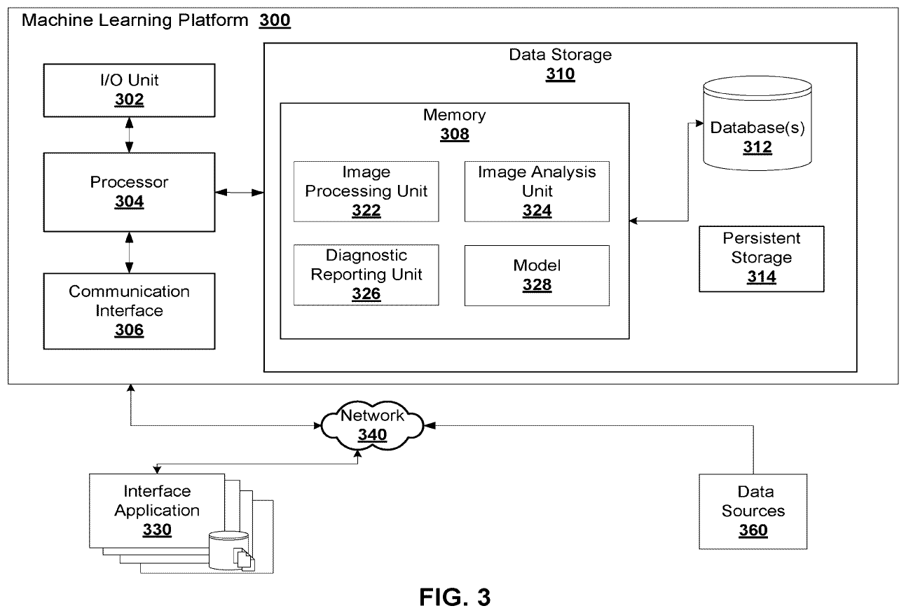

FIG. 3 illustrates, in a schematic diagram, an example of a machine learning platform 300 for at least one of estimating tissue stiffness (presented as kPa), measurements of ultrasound coefficient of attenuation, and/or characterizing tissue (e.g., liver tissue), in accordance with some embodiments. The platform 300 may be an electronic device connected to interface application 330 and data sources 360 via network 340. The platform 300 can implement aspects of the processes described herein.

The platform 300 may include a processor 304 and a memory 308 storing machine executable instructions to configure the processor 304 to receive raw ultrasonic data and/or image files (e.g., from I/O unit 302 or from data sources 360). The platform 300 can include an I/O Unit 302, communication interface 306, and data storage 310. The processor 304 can execute instructions in memory 308 to implement aspects of processes described herein.

The platform 300 may be implemented on an electronic device and can include an I/O unit 302, a processor 304, a communication interface 306, and a data storage 310. The platform 300 can connect with one or more interface applications 330 or data sources 360. This connection may be over a network 340 (or multiple networks). The platform 300 may receive and transmit data from one or more of these via I/O unit 302. When data is received, I/O unit 302 transmits the data to processor 304.

The I/O unit 302 can enable the platform 300 to interconnect with one or more input devices, such as a POCUS device, keyboard, mouse, camera, touch screen and a microphone, and/or with one or more output devices such as a display screen and a speaker.

The processor 304 can be, for example, any type of general-purpose microprocessor or microcontroller, a digital signal processing (DSP) processor, an integrated circuit, a field programmable gate array (FPGA), a reconfigurable processor, or any combination thereof.

The data storage 310 can include memory 308, database(s) 312 and persistent storage 314. Memory 308 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Data storage devices 310 can include memory 308, databases 312 (e.g., graph database), and persistent storage 314.

The communication interface 306 can enable the platform 300 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switched telephone network (PSTN), integrated services digital network (ISDN), digital subscriber line (DSL), coaxial cable, fiber optics, satellite, mobile, wireless (e.g., Wi-Fi, WiMAX), SS7 signalling network, fixed line, local area network, wide area network, and others, including any combination of these.

The platform 300 can be operable to register and authenticate users (using a login, unique identifier, and password for example) prior to providing access to applications, a local network, network resources, other networks and network security devices. The platform 300 can connect to different machines or entities.

The data storage 310 may be configured to store information associated with or created by the platform 300. Storage 310 and/or persistent storage 314 may be provided using various types of storage technologies, such as solid state drives, hard disk drives, flash memory, and may be stored in various formats, such as relational databases, non-relational databases, flat files, spreadsheets, extended markup files, etc.

The memory 308 may include an image processing unit 322, an image analysis unit 324, a diagnostic reporting unit 326, and a model 328.

The description herein will describe the ultrasound system and ML platform with reference to ultrasound images of liver tissue, estimates of stiffness, measurement of the ultrasound coefficient of attenuation, and/or identification and characterizations of liver diagnosis. It should be understood that the present description may be adopted to other tissue (thyroid, breast, kidney, prostate, bowel, pancreas, ovaries, musculoskeletal, or other organs, glands or tissues, etc.), other physical estimates or direct measurements (e.g. speed of sound, Doppler measurements, and the like), identification and/or characterizations for other diseases (e.g., inflammation, adenomas, steatosis or cancer, etc.). In some embodiments, physical estimates or direct measurements have been shown to be useful and may aid in facilitating diagnosis, screening, surveillance, triage and risk assessments, biopsy guidance, and surgical and guided interventions. Tissue characterizations identify and differentiate tissue subtypes that naturally occur in living organisms. Tissue characterizations may facilitate diagnosis, screening, surveillance, triage and risk assessments, biopsy guidance, and surgical and guided interventions.

It should be understood that while some embodiments of the ML platform involve trained models using supervised learning with predefined extracted features, other embodiments may use a deep learning model to extract non-predefined features.

### Technological Characteristics:

The proposed solution leverages and enhances technologies via a unique software and machine learning approach enabling the deployment of a safe and effective solution that will be accessible in primary care and other screening settings to provide broad availability.

The product may comprise a complete and end-to-end solution that will include a point-of-care ultrasound transducer-probe (e.g., Clarius C3) that is set and locked to the appropriate data capture configuration and a smart device with the application installed, including the proprietary AI system.

### Configuration Overview:

In some embodiments, there is provided a novel software solution that works with captured raw ultrasound signals obtained in liver scanning. Data may be acquired by an operator (i.e. clinician, nurse, technician, clinical assistant, etc.). The data acquisition is performed by protocols that optimize the capture of RF data through a series of cine sweeps that capture 10s-100s of images at predetermined locations based on liver views through abdominal landmarks (i.e. sub-coastal, intracoastal). These views are conventionally used to access the liver with ultrasound. Cine-sweeps, or cineloop sweeps are video-like sequences of a series of continuous digital images that provide dynamic views into tissues and systems. The system methods may capture hundreds of images and data points through these cine-sweeps and identifies those suitable for measuring fibrosis.

The proposed solution may comprise an application on a mobile device that provides data capture guidance and presents results to clinicians. While acquiring data through the application, the mobile screen provides a visual image of the liver and instructions to the user on which views of the liver to capture. Once the data is captured via the ultrasound system and application, the software fully encrypts the data and sends it to a secured processing system (e.g. a cloud network, a local server, an edge network, mobile communications network, or the like). The processing system may then perform a method of analysis of the raw signal data, with or without additional clinical data sets and/or biomarkers, using a specific series of AI techniques that include signal processing, quantification, machine learning and deep learning (see FIG. 2).

The application may then present the resultant score (e.g., in kPa and/or Fibrosis score), a histogram display of the score and explanation card of how the score was calculated. Further details are provided below.

In some embodiments, the ultrasound system 100 may be implemented to provide estimates of stiffness as well as measurement of the ultrasound coefficient of attenuation and in other embodiments as a liver fibrosis or a liver steatosis categorization system where the score determined is a liver fibrosis/steatosis categorization score. The system is intended to aid in the diagnosis and monitoring of adult patients with liver disease, as part of an overall assessment of the liver and/or to aid in the screening of liver fibrosis/steatosis in patients presenting a high risk of chronic liver disease. In this case, this examination looks for evidence of hepatic fibrosis/steatosis and is not intended to locate focal findings in the liver or other diffuse hepatocellular disease. Other instances and use cases can be developed via this approach to locate focal findings in the liver and/or other diffuse hepatocellular disease.

The resultant estimates of stiffness as well as measurement of the ultrasound coefficient of attenuation and/or categorization score(s), coupled with other clinical information, can be used to aid in determining appropriate intervention or referral. The device is indicated for use by healthcare professionals, including at the point-of-care.

### Process/Methodology:

In some embodiments, the system combines a series of statistical features and methods, known as radiomics, with tissue acoustic properties to establish a modeling of disease based on clinical standards Radiomic features can include first (basic histogram, mean, median, variance) and second (texture) features. Second order features specifically take into account the overall statistical relationship of one voxel to another with a single quantified measurement, and thus consider the interconnected nature of voxels through statistical means. These are good descriptors of the texture and heterogeneity of tissues, and sensitive indicators of abnormal or changing pathology of tissues through various modalities, including ultrasound.

Tissue acoustic parameters are extracted from raw ultrasound and RF data, and not through traditional B-Mode images, and are used for ultrasound-based tissue characterization (UTC). UTC parameters can be extracted through explicit and unique models, and linked through correlate analysis to unique tissue properties. These parameters provide insight into tissue microstructure, based on the frequency-based analysis of the signals from biologic tissues and other noted approaches. UTC approaches have been demonstrated to capture tissue properties in a variety of potential clinical applications that include diagnostics, surveillance and treatment monitoring in the liver, kidney, breast, and prostate.

Specific to liver fibrosis, literature evidence (further described below) has demonstrated that both radiomic and UTC parameters correlate with fibrosis grade. Commonly cited limitations to all these studies include the fact that radiomics is usually only done on B-mode images and that UTC is not done in a combined multi-parametric modeling approach.

The approach described herein builds on the established foundation of UTC for tissue characterization and removes some of the challenges of the parametric or multi-parametric approach. The approach mines the information-rich RF data using AI/ML methods to maximize on information extracted, without focusing on specific parameterization approaches. This approach allows the AI to determine the main features contained in the acoustic signal that lead to the best possible tissue characterization based on clinical-standard assessments.

The score is the output of an AI/ML subsystem that has demonstrated accuracy against an accepted standard for fibrosis staging. In some embodiments, the AI subsystem has been trained on over 2,000,000 data points from over 4,000 individual (see FIG. 2) patients determined to be normal or having fibrotic or cirrhotic tissues. The system has initially been trained using “stiffness” measures generated by transient elastography systems and measured in kilopascals (kPas), combined with demographics and the documented and underlying chronic liver disease, to classify the expected level of fibrosis which may be present. Currently, the FibroScan system is a clinical standard for detection and hepatology-based diagnosis of fibrosis. Thus, the FibroScan Fibrosis Staging System in combination with patient demographics and CLD, with other confirmed diagnosis by other imaging modalities or biopsy when available, may be used to provide ground truth for training purposes.

### Data Input:

In some embodiments, the system designates liver fibrosis based on tissue characterization properties as determined by ultrasonic radio frequency (RF) signals used to measure tissue acoustic properties. The software registers RF data and performs computer analytics based on quantitative ultrasound (QUS) and radiomic parameters. The data retrieval using a transducer (e.g, a Clarius transducer) is capable of scanning cross-sectional views of the liver, and is estimated to capture up to ˜80% of a patient's liver tissue depending on body mass index (BMI). The data collection has been tested on patients with a range of BMIs up to 47. Beyond the main RF data, in some embodiments the system may use as inputs other relevant clinical data such as demographic data (e.g. gender, ethnicity, race, etc), clinical data (height, weight, prior and/or underlying conditions, etc.), biomarker and fluid data (e.g. blood or excreta test results, saliva, etc.).

### Liver Access Points:

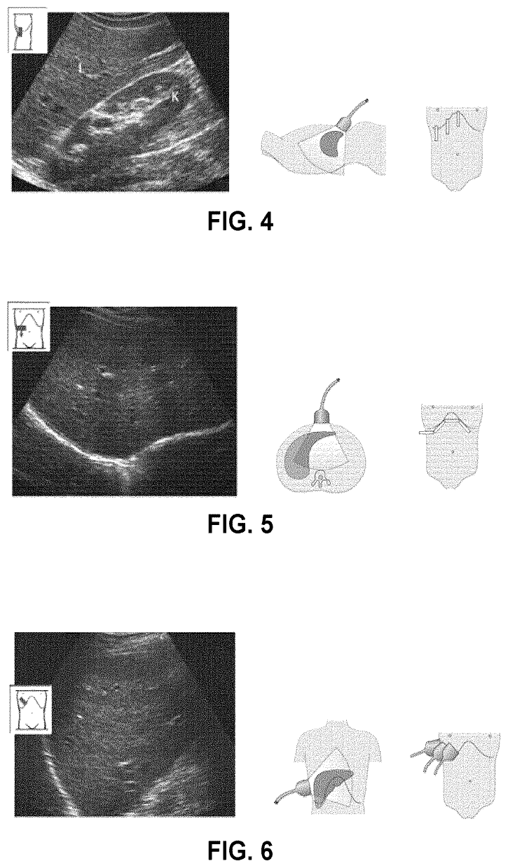

FIGS. 4 to 6 illustrate an ultrasound image and a corresponding scan angle, in accordance with some embodiments. FIG. 4 shows a subcostal sagittal (SCS)/subcostal sagittal mid-axillary line (SCS-MAL) angle. FIG. 5 shows a subcostal transverse (SCT) angle. FIG. 6 shows an intercostal space (ICS) angle. In some embodiments, the AI/ML subsystem was trained on ultrasound frames taken from four (4) major scan angles listed below. The large majority of frames, which capture maximum liver tissue in the collected training data, come from the subcostal and intercostal viewpoints. The intercostal access points are in-between ribs in the lowest portion of the ribcage (2 most caudal spaces).

### System Ultrasound Scan Angles (See FIGS. 4 to 6):

IC1: Intercostal Space 1 (most caudal)
IC2: Intercostal Space 2 (next most caudal)
SCS MAL: Subcostal side mid-axillary line
SCT: Subcostal Transverse

The liver has four main lobes: the large right lobe, a small left lobe, the caudate lobe and the quadrate lobe. In patients with liver fibrosis, the right lobe is more fibrotic than the caudate lobe, and in cases of cirrhosis, atrophy is visible primarily in the right lobe. The earliest region that develops detectable fibrosis is usually in anterior segments V and VIII of the right lobe of the liver. These segments are accessible through SCT and ICS liver scan angles, which are required in abdominal sonography protocols. The intercostal and subcostal scan planes effectively capture tissue in the large right lobe of the liver, and are the primary scans required for the system exam.

If there is poor visibility of the liver, the patient is asked to hold a deep breath, expanding the lungs and effectively pushing the liver caudally, causing large portions to be accessible for subcostal visualization. Additionally, patients may be asked to raise their right arm above the head to draw the rib cage upwards. If the subcostal scan is difficult due to impenetrable gas, the intercostal scan angles typically capture liver tissue well.

Requiring both subcostal and intercostal scan planes ensures that several frames of liver tissue will be captured within the system data acquisition videos. Scan planes required in the system are consistent with the scan planes captured in the training data used to develop the system algorithm. Practices such as asking patients to hold their breath, or raise their right arm were common during data collection to improve liver visibility. These practices are also encouraged in the system application, and may be incorporated in system training sessions.

### Ultrasound Data Collection:

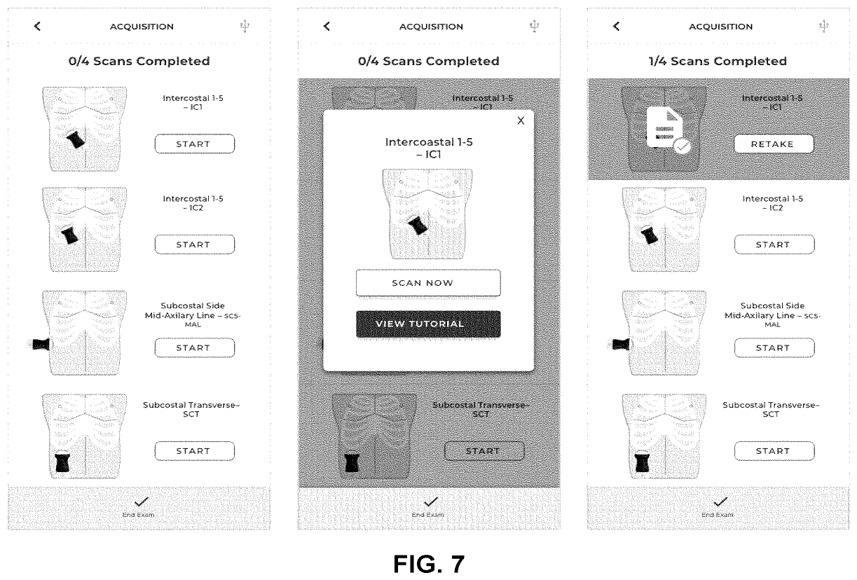

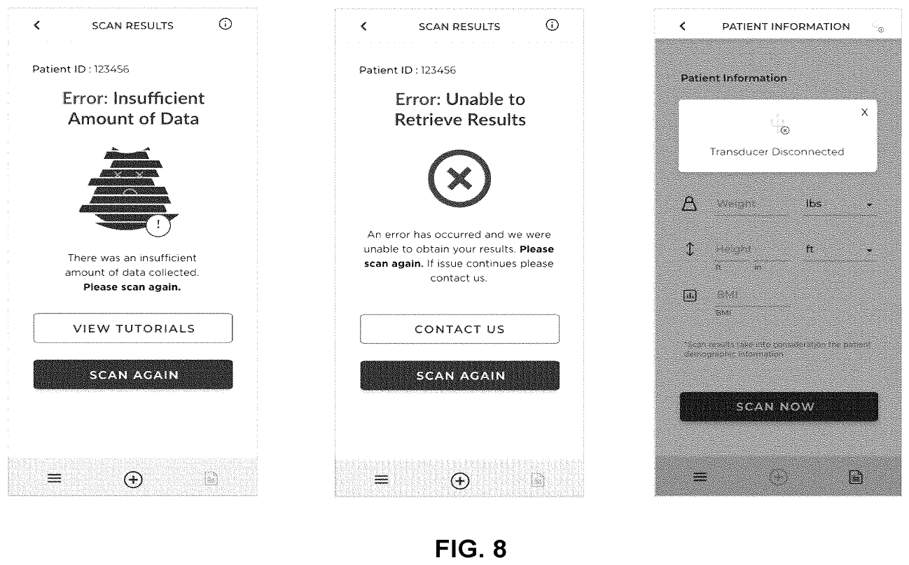

FIGS. 7 and 8 illustrate, in screen shots, examples of data acquisition 700 and scan results 800, in accordance with some embodiments. The system includes a data collection protocol that facilitates accurate data collection for each patient and the end user is not expected to have had any prior ultrasound training beyond the supplied training. The system instructs the clinician to capture certain views of the liver (subcostal or intercostal) while the patient is holding their breath and marks the views as complete once they are acquired (see FIG. 7). The acquisition guidance advises the user on which categories of views to collect. It also performs a usability and quality check on the data and only returns a score when sufficient data has been captured. (see FIG. 8). The sweeps of data at the specified locations allow the system to amalgamate large data sets that are then sorted and segmented. In some embodiments, the data is acquired with the same pre-sets to ensure absolute and comparable quantification. To enhance accuracy and ease of use, an automated method of liver segmentation (described below) ensures that only liver tissue is analyzed by the AI subsystem. Additionally, a method of RF signal detection (described below) may also provide feedback to the clinician, regarding the level of shadowing and other impediments, which may obstruct signal quality. As described in further detail below, the system provides feedback to the user if insufficient signal data is collected. For example, if not enough data is collected for the system Score, the system returns “Error: Insufficient Amount of Data.” Should other errors occur during app process, the system returns “Error: Unable to Retrieve Results.” The system can also provide updates to users based on their connection with the transducer. In some embodiments, the system may provide feedback to the user to retake one or more scans if, for example, insufficient data is collected. In some embodiments, a user interface may notify the user that they should re-take one or more scans.

### Liver Detection and Segmentation:

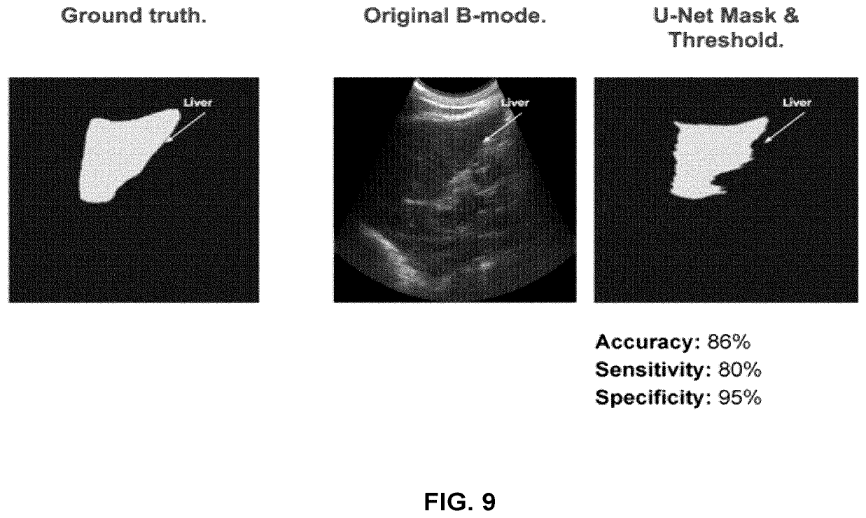

FIG. 9 illustrates an example of a liver segmentation model 900, in accordance with some embodiments. The model may be used to identify liver tissue within the 100s of image-data obtained for each patient. This model is iteratively improving as segmented data is added. After achieving desired specificity and sensitivity and before release, the model and AI algorithm may be locked.

In some embodiments, the system automatically segments liver tissues to focus the feature extraction and analysis on that data only. The segmentation algorithm may be trained from images labeled by professionals and quality control reviewed by sonographers and radiologists. The algorithm may use a U-net neural network (see FIG. 9). The segmentation algorithms perform two functions: 1. Identifying the liver tissues; and 2. Identifying which frames have enough liver coverage to be usable. The system may include a set minimum threshold of identifiable liver in a frame in order for the frame to be considered usable in the ML assessment. Further details are provided below.

### RF Signal Quality Detection:

Prior to processing the data, the system may detect frames with significant RF signal void, indicative of acoustic shadowing or other artefacts. Frames may be marked with a percentage of signal void and compared to a pre-set threshold. The system then removes any frames that surpass this threshold to enhance the specificity of the signals being processed. The system provides feedback to the clinical user if the scan does not have adequate coverage, as displayed in FIG. 8.

### Statistical Summaries:

The system builds on UTC parameters and combines these in a novel manner to provide a clinically meaningful tool (see Table 1). The statistical summaries and parameters used have been shown in research to be effective at tissue characterization.

TABLE 1
UTC acoustic property features used in the system

| Method | Parameter(s) | Evidence |
|---|---|---|
| Back Scatter Based Methods, | Backscatter coefficient | Many have reported the |
| related to the power spectrum and | (BSC), mid-bandfit (MBF), | effectiveness of these parameters |
| energy of the reflected signal. | Spectral Slope (SS), | in Tissue Characteristics. |
| Considered the basis of | Spectral Intercept (SI) | |
| Quantitative Ultrasound | | |
| Techniques. | | |
| Statistical method of analysing the | NS (Nakagami Spread), | Shown utility in identifying |
| distribution of reflected signal, as | NSH (Nakagami Shape) | NAFLD and steatosis. |
| reflected RF does not follow a | | |
| normal distribution. | | |
| Backscatter based method, | ESD (effective scatterer | Shown to be effective for liver |
| related to the shape of the | diameter), EAC (effective | fibrosis and other cancer-based |
| reflected signal. | acoustic concentration) | UTC. |
| Measurement of rate of energy | AC (Attenuation | Increasingly shown to be clinically |
| loss through different mediums. | Coefficient) | relevant for steatosis and other |
| | | forms of UTC. |

### Machine Learning:

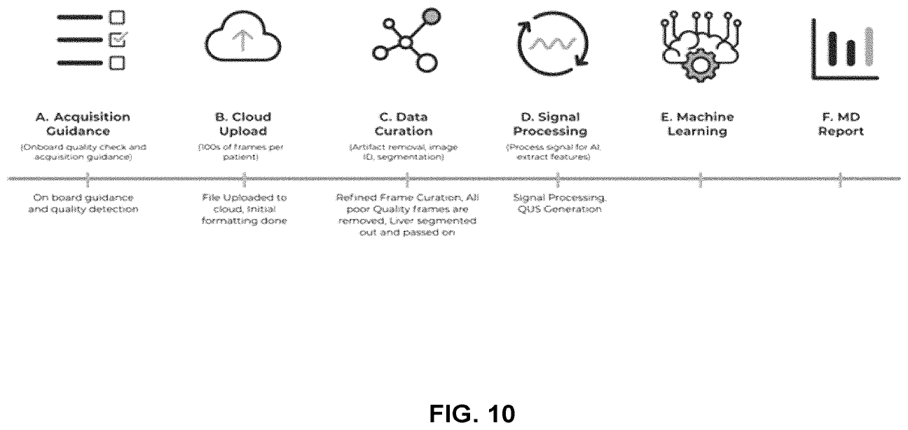

After all quality checks and statistical summaries are processed, the resultant data may then be processed using a machine learning method. In some embodiments, the machine learning method may use 10-fold cross-validation. FIG. 10 illustrates, in a timeline diagram, an example of a processing pipeline 1000, in accordance with some embodiments. The algorithm processes each frame, from the tens to hundreds of frames available per patient, as described in FIG. 10. In steps A-F, several UTC features are extracted (see Table 1) on a frame level using second order statistics, and summarized per patient to produce patient-level features to classify each data point using machine learning. Each set of patient data is then compared to the library of accrued liver data that has been labelled with stiffness measures in kilopascals (kPa), demographic data including underlying CLD when known, as well as any other confirmed diagnosis (e.g. Alcoholism, HIV, etc.).

At step A acquisition check system, the system instructs the user to capture certain views of the liver (subcostal or intercostal while the patient is holding their breath), and marks the views as complete once they are acquired. In some embodiments, the system may inform or otherwise provide feedback to the user that one or more scans need to be retaken (if, for example, insufficient signal data is collected).

At step B cloud upload, once acquisition is complete, the videos may be submitted to be uploaded to the cloud for analysis.

At step C data curation, the videos and frames are then segmented and filtered to ensure only quality liver portions of the scans are passed onto the next step. Frames with adequate liver tissue are used for feature extraction.

At step D signal processing, UTC features are extracted (see Table 1). Texture features are calculated from maps of UTC features. Both feature sets may then be processed via the ML. In some embodiments, additional data inputs may be used, including but not limited to clinical data sets and/or biomarkers, tissue stiffness, and ultrasound coefficients of attenuation.

At step E machine learning, the features are fed into the ML subsystem, and the Score is determined.

At step F MD report, the score, along with other information generated, is synthesised into a comprehensive report, which is sent back to the application on the user device for the MD to evaluate.

### Sample Output:

Under the use case of diagnosing and staging liver fibrosis, the device provides a score based on comparative analysis to liver ultrasounds with known diagnoses output in a structured report with a histogram display available. The score is based on a machine-learning algorithm, trained on a subset of features. In some embodiments, a single value score, a score of 1-10, is a summary of ML and signal processing, relating the scan to other liver scans with similar features. In some embodiments, the number relates that patient to different normative patient set within a library. For example, the proportion of Normal (F0-F1, little to no fibrosis), Fibrotic (F2/F3), and Cirrhotic (F4) patients in the OnX “number group” may be displayed as percentages. In some embodiments, an example output may be that X₁% were normal (i.e. had normal scores), X₂% were fibrotic and X₃% were cirrhotic and then use this to inform their clinical decisions. In some embodiments, an output may include a resultant stiffness estimate in kPa ranges and/or an attenuation measurement as a discrete number.

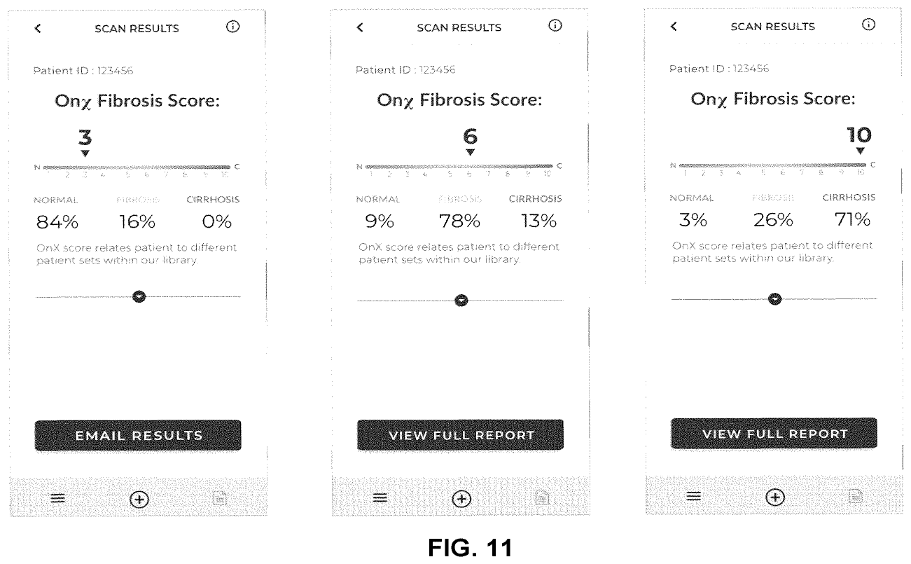

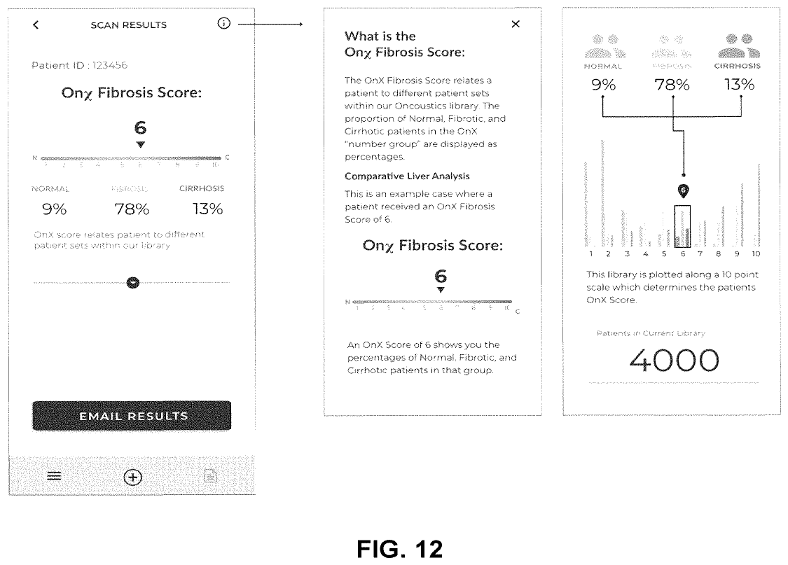

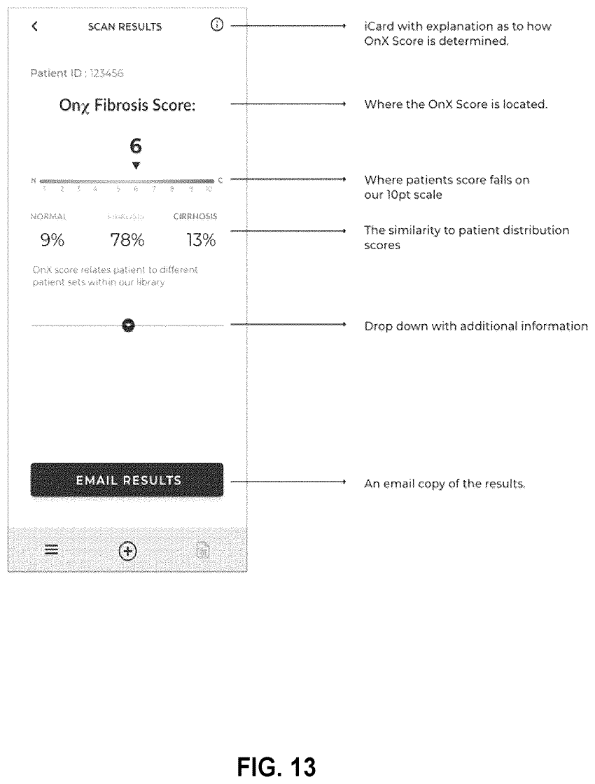

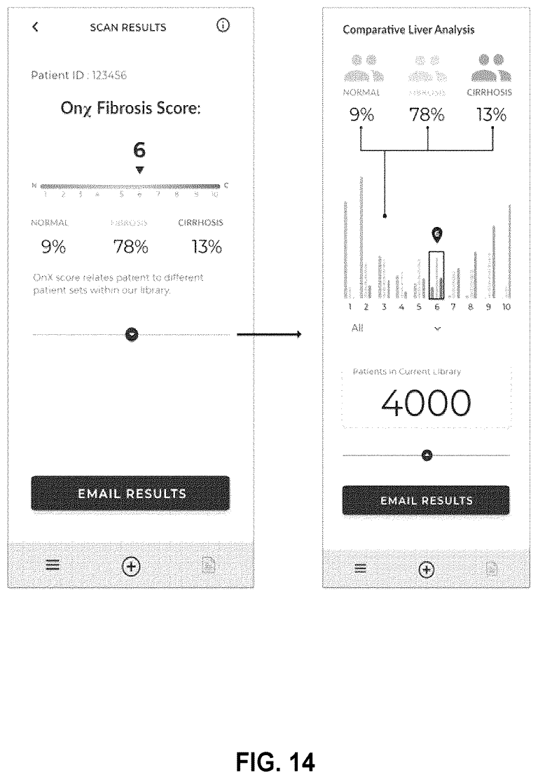

FIGS. 11 to 14 illustrate examples of the initial results page, in accordance with some embodiments. FIG. 11 shows examples of results page displaying the score and the proportion of normal, fibrotic and cirrhotic patients. FIG. 12 shows a results page with an information card which explains to the user how the score is determined. FIG. 13 shows the breakdown of information provided on the application screen including the Fibrosis Score, and the similarity to patient distribution. FIG. 14 shows that by clicking on the additional information drop down, users can view a histogram that relates that patient to different patient sets within the library, the proportion of Normal, Fibrotic, (and possibly Cirrhotic) patients in each “number group” is displayed as a graph on the report. In some embodiments, users can use drop down menus to change the Population that this graph displays. In some embodiments, the number of patients in the system's current library may also be displayed.

In some embodiments, the user can also view additional information with a set of histogram graphs representing all 10 scores and the proportions of Normal, Fibrotic and Cirrhotic patients. The score is intended for the organization of an online atlas (reference database) provided to the clinical user as the Similar Case Database. Patient numbers are represented as a proportion of the overall class to which they belong.

### Feasibility Data

### Data Collection:

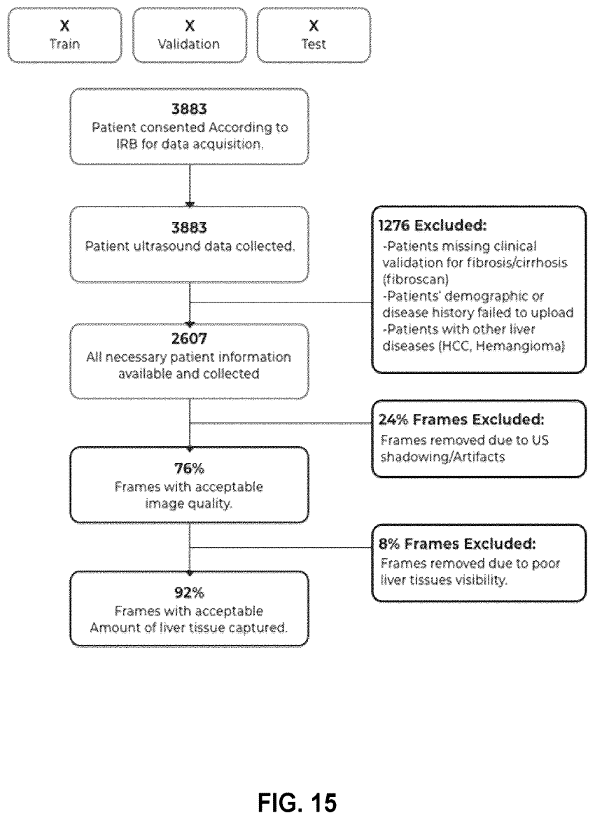

In some embodiments, the system includes a unique set of raw RF data for UTC analysis in patients at risk for or with known liver disease receiving a clinical standard assessment for fibrosis. Data acquisition efforts are broad and have been undertaken at five public clinics including international and domestic clinics. Patient data represents a wide range of ages, underlying chronic liver diseases that may result in fibrosis/cirrhosis and body mass indices (BMI) with successful scans taken in patients with BMI of up to 47. This represents an effort to assemble a large library of raw RF ultrasound data with clinical standard assessments of liver disease (see FIG. 15). Our IRB-approved protocols and systems are designed to facilitate data acquisition through a series of sweeps that can be performed with ease. The sweeps capture liver tissue through the main abdominal access points, and store hundreds of RF-based ultrasound frames through cine sweeps.

### Training Data Distribution:

FIG. 15 illustrates, in a data flow diagram, an example of a method of data collection enrolment 1500, in accordance with some embodiments. In some embodiments, the system RF data library comprises patients with a range of documented and relevant chronic liver disease histories (HBV, HCV, Alcoholism, Steatosis, Obesity, etc.) that may result in fibrosis. In addition, other key patient demographic data is acquired. The system relies on this data to train a machine learning subsystem to algorithmically assemble UTC features in such as a way as to differentiate between normal (F0/F1) and fibrotic (>=F2) tissues, based on the METAVIR scale.

The METAVIR fibrosis score is used to describe the amount of fibrosis in the liver:
F0: No fibrosis
F1: Portal fibrosis without septa
F2: Portal fibrosis with few septa
F3: Numerous septa without cirrhosis
F4: Cirrhosis

Labels for the system model may be based on clinical interpretation of FibroScan results in conjunction with demographic/history information. For example, the FDA has cleared the Fibroscan for the following indication: “FibroScan® is intended to provide 50 Hz shear wave velocity measurements through internal structures of the body. FibroScan® is indicated for noninvasive measurement of shear wave speed at 50 Hz in the liver. The shear wave speed may be used as an aid to clinical management of patients with liver disease.” Thus, while Fibroscan is not cleared by FDA for use as a screening tool, it is an appropriate tool for measuring liver stiffness (kPa), and is capable of staging liver fibrosis according to the METAVIR scoring system. Cut-off values in the system classify F0-F1, F2, F3, and F4 fibrosis which vary based on concurring liver diseases (i.e. Chronic Hepatitis B). For each patient data set, fibrosis staging is further confirmed by hepatologists and through published relationships between the METAVIR scale and kPa range.

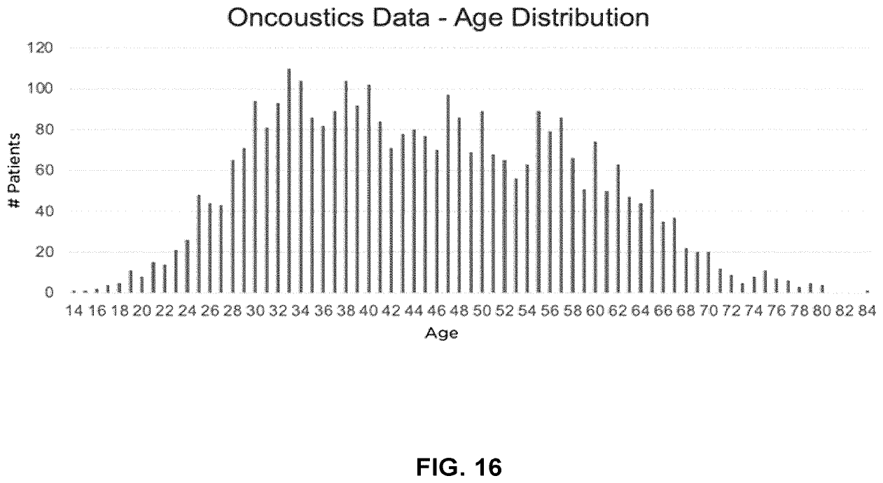

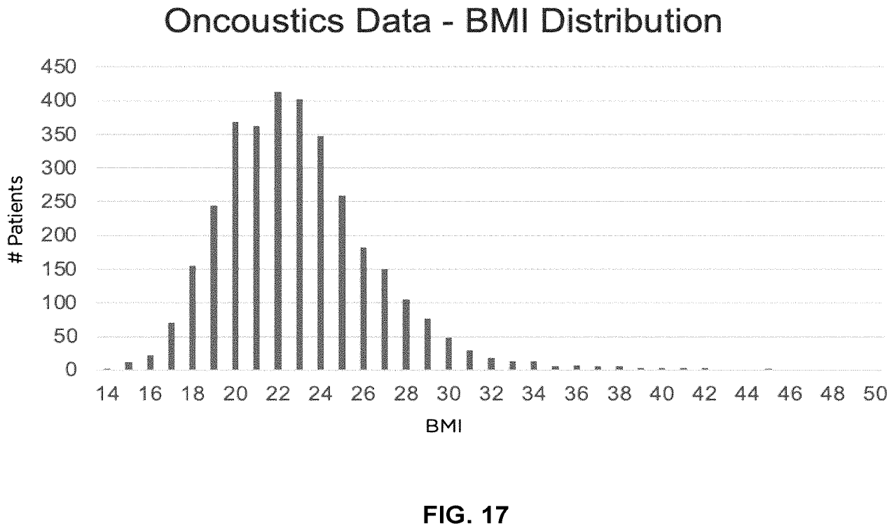

The liver fibrosis categorization system may be used to aid in the screening of liver fibrosis intended to support the detection and categorization of fibrosis in any patient presenting a risk of fibrosis from a confirmed or suspected chronic liver disease. FIGS. 16 and 17 illustrates distributions of Age and BMI in the patient data set used to develop and test the models, in accordance with some embodiments. FIGS. 16 and 17 display histograms of patient distributions in the system library used to test the system machine learning models, including those in the reserve data sets. Patients in reserve data sets were not used to train any of the models, and are therefore viable points of comparison to any incoming patient that are received. FIG. 15 shows the distribution of patient BMIs in the system training, validation and testing datasets.

### Machine Learning Best Practices:

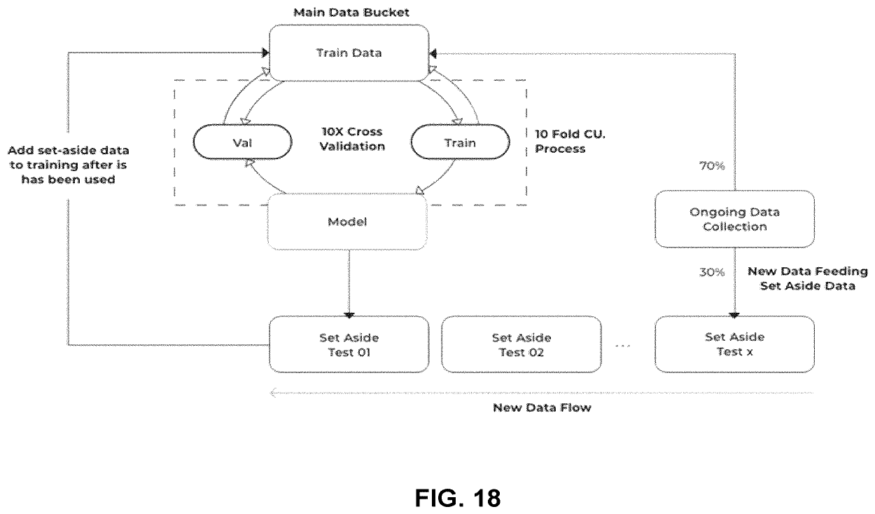

Machine learning training is carried out using 10-fold cross-validation methods, as well as set aside data sets that are not tested and preserved until a certain phase of training is achieved; new set-aside data is continuously being prepared as we accrue more data. Further details on the current set-aside buckets are presented in FIG. 18.

FIG. 18 illustrates an example of ML training, in accordance with some embodiments. Machine learning training is carried out using 10-fold cross-validation methods. Randomly selected data sets are placed in a “set aside” that are that are not tested and preserved until a certain phase of training is achieved. These are then used for ongoing assessment and validation. New set-aside data is continuously being prepared as more data is accrued. During each period, 70% of the new patients were added to the train set, and 30% were placed in reserve buckets. This is the continuous process maintained to ensure adequate validation of the models.

### Performance Measurements:

Through the system's ongoing data collection, new set-aside data buckets may be continuously created for validating the algorithm, in addition to traditional cross-validation methods. The train, cross-validation, and set-aside testing methodology is described in FIG. 18. In brief, as new buckets of set-aside data (˜100 patient cases per bucket) are created, the most recent and improved models may be tested before moving this data to be part of the training bucket. This process may be repeated with new set-asides every few weeks to months.

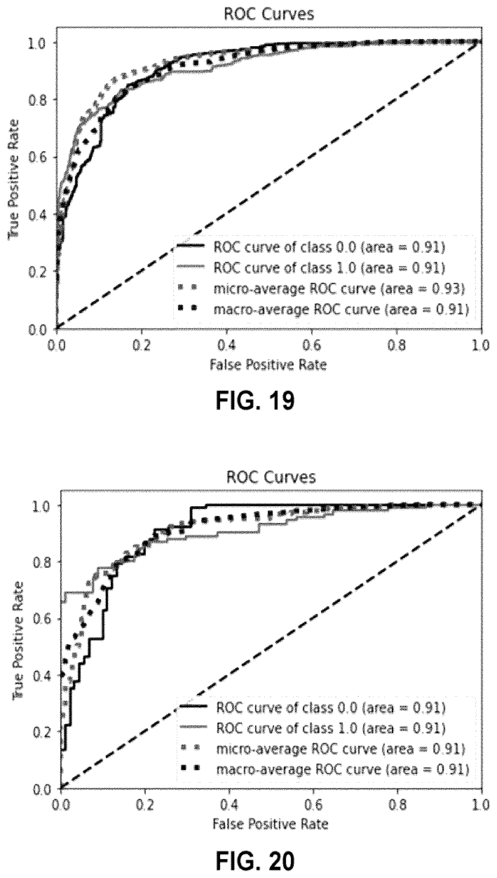

For cross-validation, the average system receiver operator curve area under the curve (ROC-AUC) for all 5 folds was 89% (+/−4%) compared to a fibrosis assessment done via FibroScan. This evaluation metric is expected to further improve as additional data is acquired before locking the system for clinical release. Results with key machine learning metrics (accuracy, F1 score, sensitivity, specificity) with an optimal threshold are presented in FIGS. 19 and 20. Note that this is amongst the initial iterations of the algorithm, and it is expected to achieve an AUC >95% and an accuracy >90% before locking the data/models and obtaining regulatory clearance. FIG. 19 illustrates, in a graph, an example of a model ROC for training data set, in accordance with some embodiments. FIG. 20 illustrates, in a graph, an example of reserve data set ROC curves, in accordance with some embodiments.

TABLE 2
Model performance on training data set

| | True Positive | True Negative | |
|---|---|---|---|
| Predicted Positive | 316 | 52 | f1: 0.78 |
| Predicted Negative | 139 | 882 | TNR: 0.94 |
| | Precision: 0.86 | Recall: 0.7 | Accuracy: 0.867 |

The system may hold several set-aside buckets for future evaluations (see FIG. 18 for details). In some embodiments, the system holds 4 set-aside data buckets, which were obtained through continuous prospective acquisition at clinics, within the following periods: from May 2019-June 2020, June 2020-July 2020, July 2020-November 2020, and November 2020-February 2021. The most recently developed model was evaluated on a set aside of 181 patients imaged from May 2019 to June 2020. The ROC-AUC for this set-aside data is 89%; additional key metrics are presented in FIG. 20 and Table 3. As presented in the cross-validation results, the algorithm is capable of assessing fibrosis with high sensitivity and specificity.

TABLE 3
Confusion matrix for model evaluation on set-aside #1

| | True Positive | True Negative | |
|---|---|---|---|
| Predicted Positive | 70 | 9 | f1: 0.83 |
| Predicted Negative | 20 | 82 | TNR: 0.96 |
| | Precision: 0.89 | Recall: 0.78 | Accuracy: 0.840 |

Compared to the current standard of care that is available in primary care or at point of care, the system may enhance the ability to detect fibrosis at all stages and shows a significant improvement in accuracy over existing tools.

TABLE 4
Sensitivity, Specificity, Accuracy and Fibrosis Stage comparison

| Type of | | | | Fibrosis | |
|---|---|---|---|---|---|
| Test | Sensitivity | Specificity | Accuracy | Stage | Notes |
| Blood Tests | ~86 | 55-65 | 60-70 | F0/F1 - | Large |
| | | | | from F3+ | variability on |
| | | | | | particular test |
| | | | | | and cohort |
| | | | | | Results vary |
| | | | | | drastically |
| | | | | | based on |
| | | | | | cutoff. |
| Blood Based | 72 | 87 | 75 | F0/F1 - | Large |
| Biomarkers | (range | (65-92) | (63-80) | from F3+ | variability |
| | 63-86) | | | | between test |
| | | | | | and cohort |
| | | | | | Results vary |
| | | | | | drastically |
| | | | | | based on |
| | | | | | cutoff. |
| OnX | 78 | 90 | 87 | F0/F1 - | Enhanced ability |
| | | | | from - F2+ | to detect |
| | | | | | fibrosis at |
| | | | | | all stages |
| | | | | | Optimal |
| | | | | | threshold |
| | | | | | determined |
| | | | | | from training |
| | | | | | data. |

In some embodiments, the teachings herein describe a device that provides for more effective treatment or diagnosis of life threatening or irreversibly debilitating human disease or condition. In some embodiments, the system may provide early and enhanced risk assessment and stratification, with the ultimate goal to drive early interventions in a growing population who are or will be affected by silent liver diseases that, undetected and untreated, ultimately lead to increased morbidity and early death.

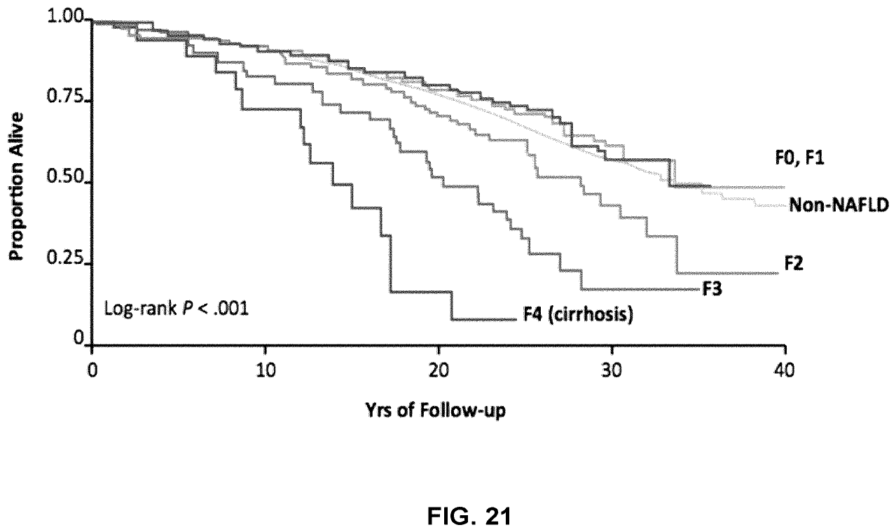

Regardless of the type of underlying liver disease, fibrosis stage and progression rate are both quintessential determinants of liver health, and patient morbidity and mortality risks. Several studies and experts have indicated that the degree of fibrosis is the key prognostic indicator in patients with or at risk of liver disease. The risk of liver-related mortality increases significantly with increase in fibrosis stage. (see FIG. 21)

FIG. 21 illustrates, in a graph, an example of survival analysis of patients, in accordance with some environments. Liver Fibrosis Is a Risk for Adverse Outcomes. Retrospective survival analysis of 646 NAFLD patients and matched controls.

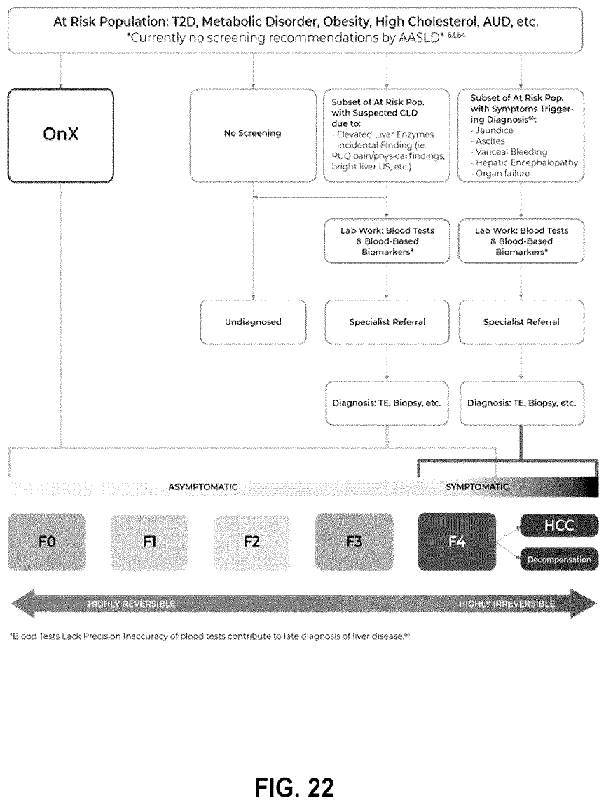

FIG. 22 illustrates an example of a system stage shift vs the standard of care for high risk patients, in accordance with some embodiments.

| | MANY PATIENTS ARE DIAGNOSED |
|---|---|
| FIBROSIS IS OFTEN UNDIAGNOSED | TOO LATE WITH SYMPTOMS |
| A prospective screening study of T2D | Cirrhosis was unrecognized until the |
| patients without any known liver disease, | development of HCC in 64% of patients with |
| 7.1% had undiagnosed advanced fibrosis. | a clear indication for screening. |
| A large prospective study recruited 1118 | Only 1 in 3 people with cirrhosis know that |
| patients in the primary setting with incidental | they have it. |
| abnormal LFTs and found undiagnosed | In a US population based study, NAFLD |
| advanced fibrosis in 7.6% of patients. | was the most common underlying etiologic |
| In a cohort of 1918 patients with T2D without | risk factor for HCC (59%), and increases |
| any known liver disease, 2.3% were found to | with advances liver disease. |
| have undiagnosed advanced fibrosis or | Cirrhosis is the 12th leading cause of |
| cirrhosis due to NAFLD. | death in the United States. |
| A retrospective study showed that 63.9% of | |
| patients undergoing | |
| pancreaticoduodenectomy had undiagnosed | |
| liver fibrosis. | |
| Fibrosis is significantly underdiagnosed by | |
| diabetes specialists. | |
| Widespread liver disease is missed in | |
| patients with T2D. | |

Early detection and monitoring of liver fibrosis in high-risk populations can serve as a prognostic indicator of overall liver health and underlying chronic liver disease progression. In order to minimize fibrosis-related complications such as cirrhosis, cancer, transplant or death, increasing numbers of key-opinion leaders (KOL) have repeatedly called for early fibrosis detection, screening and continuous monitoring outside of hepatology, within relevant subspecialties (i.e. endocrinology and cardiology) or in primary care. As such, given the high prevalence and resulting mortality of cirrhosis, fibrosis should be detected, characterized and monitored in early stages in high-risk populations, when possible.

### Liver Fibrosis Categorization System:

Many groups and organizations, including the FDA in its published draft industry guidance, Noncirrhotic Nonalcoholic Steatohepatitis With Liver Fibrosis: Developing Drugs for Treatment Guidance for Industry (2018), have called for non-invasive biomarkers to accurately diagnose and assess various grades of NASH and stages of liver fibrosis. In addition, there is a clear need for a cost-effective, non-invasive and accessible screening tool to be used by primary care physicians (PCPs) during routine patient appointments, to assess the risk of fibrosis and determine whether a patient should be sent to a specialist. The system comprises a complete solution that leverages inexpensive and widely available portable ultrasound hardware devices, and augments those units with machine-learning-based quantification of tissue acoustic signatures that correlate to tissue characteristics such as fibrosis and fibrosis stage. With a simplified 5-10-minute liver scan, with minimal training (see Feasibility section: Training above) a clinician can receive results from an AI algorithm, which presents the likelihood of fibrosis and cirrhosis by comparing a patient's liver to an existing ultrasound database of >4000 patients.

In some embodiments, based on the system models, the system categorizes liver fibrosis with a ˜91% Area Under Receiver Operating Curve (AUC) and ˜87% accuracy against current clinical standards for measuring fibrosis in tertiary specialty care. Compared to the current standard of care that is available in primary care or at point of care, including blood tests and new blood based biomarker algorithms, the system enhances the ability to detect fibrosis at all stages and shows a significant improvement in accuracy over existing tools through a five-minute office exam.

By expanding access to non-invasive fibrosis staging, the system has the potential to improve the diagnosis of liver fibrosis, a debilitating and potentially life-threatening condition, allowing for more timely intervention and improved treatment outcomes. Therefore, the system satisfies the first criterion for Breakthrough Status.

In some embodiments, the teachings herein describe a system that characterizes tissue structural and physical properties via point-of-care ultrasound devices. The system leverages tissue acoustic properties present in raw radio-frequency (RF) ultrasound data (conventionally discarded after an ultrasound image is formed), and linking those acoustic properties to tissue characteristics of interest using AI (i.e. fibrotic vs. normal tissues).

In some embodiments, the system approach may combine the RF signal and image data, with or without additional clinical data sets and biomarkers, and may pass this all through multiple processing algorithms including ones for liver segmentation, automated artefact detection and data usability checks, acoustic feature extraction and machine learning to provide quantitative and comparative assessment of a patient's liver.

As the system approach does not rely on image quality for any qualitative assessments, relying instead on the raw sound signals, the system can be deployed on low cost, handheld point-of-care ultrasound systems that currently have limited medical value due to poor image quality. These low cost systems combined with the unique approach, enables the system to expand access to liver surveillance screening to point-of-care, bedside or in remote settings.

Furthermore, as the system approach can utilize as ground truth any and all other diagnostic tests or combinations of these tests, the technology can be expansive and the performance of the resultant system has the potential to be superior to any diagnostic currently available.

In some embodiments, the teachings herein describe a device that offers benefits over existing approved or cleared alternatives, including the potential, compared to existing approved alternatives, to reduce or eliminate the need for hospitalization, improve patient quality of life, facilitate patients' ability to manage their own care, or establish long term clinical efficiencies.

The system facilitates early diagnosis of structural liver disease at point-of-care, which can—if caught early enough—be managed by the patients and their care teams outside of hospital settings and can even be reversed. Compared to available alternatives, the system is also relatively fast, painless, non-invasive and with no radiation exposure. And this test, which can be completed and have data shared with the patient within the span of a single point-of-care clinical visit without the need for referrals or unnecessary second visits is a more clinically efficient solution for patients and their care teams alike.

As noted, traditional liver function tests that measure the levels of certain enzymes and proteins in the blood are notably poor at screening for fibrosis or cirrhosis. New serum combination tests, while improvements over standard liver enzyme tests, have been shown to have significant limitations such as variability, inadequate accuracy and significant limitations with respect to sensitivity and specificity. Liver blood tests have remained almost unchanged since they were developed in the 1950s, with the result being that many patients with liver disease are not identified until they have developed significant liver fibrosis. Compared to the current standard of care that is available in primary care or at point of care, the system enhances the ability to detect fibrosis at all stages and shows a significant improvement in accuracy over existing tools. (See Table 4 and FIG. 22).

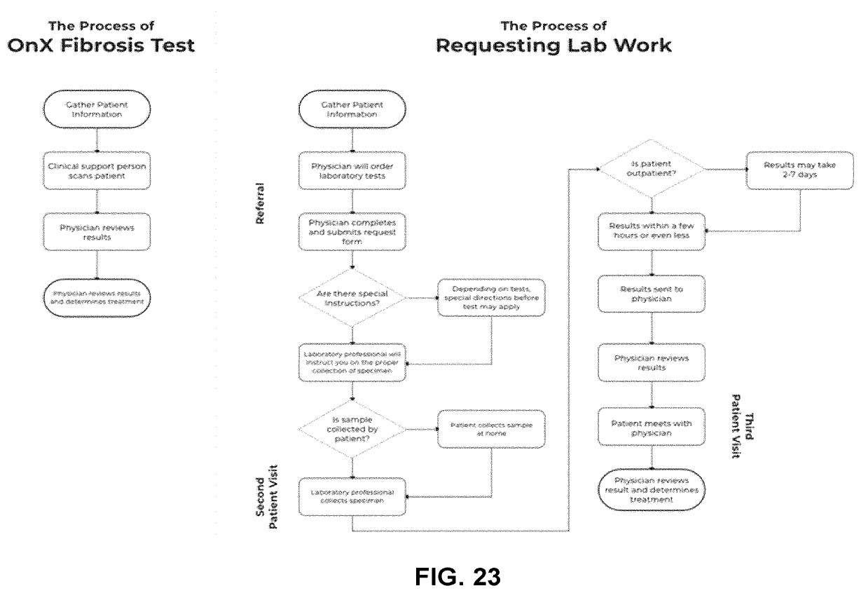

The Liver Fibrosis Categorization and/or Liver Assessment Solution systems exams can be completed and have data shared with the patient within the span of a single clinical visit. In a currently know blood panel, the patient needs to go to a blood draw lab or to a trained phlebotomist, wait for pathology to review and then the clinician to relay the findings (see FIG. 4). Other types of known specialty testing require even more steps, costs and friction for the patient, as well as clinical inefficiencies for the clinician. Patients benefit from being able to access an important diagnostic screening test at point of care, rather than being referred to a specialist or a centralized hospital system. This access and availability removes the friction of several appointments, traveling to referral centers, etc. for patients and decreases both direct and indirect costs (see FIG. 23).

FIG. 23 illustrates, in process flowcharts, a comparison of a system fibrosis test vs a process of requesting lab work. The system Fibrosis Test improves the patient experience, makes tests more accessible, and establishes long-term clinical efficiencies by enabling front line physicians to easily perform tests in the clinic and/or at the bedside rather than needing to refer the patient elsewhere or transport to radiology.

### The Liver Fibrosis Categorization System:

By comparison, the system works on low-end, inexpensive (˜$2,000-6,000) and widely available point-of-care ultrasound systems connected to a smart device. The test can be performed in 5-10 minutes via a series of fast, painless and visible ultrasound cine-sweeps over the abdomen that anyone can acquire with minimal training. The system approach uses the kPa scores, with underlying and documented CLD, other documented relevant disease history (HIV, alcoholism, T2D), patient demographic data (age, weight, etc.) and other confirmed diagnostics data (MR-PDFF or biopsy) when available, to serve as ground truth and train the AI. The results and degree of fibrosis are presented to the clinicians as an ‘OnX’ numbered score on a fixed-point scale. In some embodiments, the scale is a 10 point scale. The clear and quantitative patient evaluation can help point-of-care clinicians determine patient risk and make referrals to specialists if deemed appropriate.

In sum, the system facilitates early disease detection, makes tests more accessible, improves the patient experience and establishes long-term clinical efficiencies by enabling front line physicians to easily perform tests in the clinic rather than needing to refer the patient elsewhere or transport to radiology (See Table 5).

TABLE 5
Table compares existing liver tests with the OnX Liver Fibrosis Categorization Test

| Type of Test | Overview | Objective | Benefits | Issues and Concerns |
|---|---|---|---|---|
| Hepatic | Blood tests which | Measures liver | Inexpensive. | Requires lab visits for blood |
| Panel | typically include | enzymes and | Can be ordered | draw. |
| | measurement of | normal liver | by a POP at | Interpretation can take 2-7 |
| | albumin, ALT, | functions in | PoC. | days for outpatients. |
| | AST, ALP, | producing protein | | Test results do not always |
| | Bilirubin and | and clearing | | correlate with disease level. |
| | Prothrombin time. | bilirubin. No | | Tests can be normal even |
| | Other tests may | predictive value. | | with advanced liver |
| | also be included. | | | disease. |
| Blood Test + | Application of | Adds objective | May have | Suited to ruling out |
| Algorithm | proprietary | measures that may | additional | advanced disease and for |
| 1. Fibrosure | algorithms to | have predictive | negative | its negative predictive |
| 2. FIB- 4 | blood results and | value to better | predictive | value. |
| 3. NIS- 4 | other factors (age, | assess patients | value. | Does not offer staging and |
| 4. LIVERFast | BMI, etc.) to | over simple blood | | cannot rule out advanced |
| | assess liver | tests alone. | | liver disease. |
| | health. Specific | | | Tests can be expensive |
| | blood results used | | | and require specialty labs |
| | varies by | | | and significant |
| | algorithms. (See | | | interpretation. |
| | examples.) | | | |
| Transient | An ultrasound | Produces a score | Noninvasive. | Generally prescribed when |
| Elastography/ | device that | used to assess | Offers confirmation | there is an existing |
| Fibroscan | measures the | liver stiffness that | of advanced | indication of liver damage. |
| | velocity of sound | can be correlated | disease. | Limited predictive value. |
| | waves which | with liver fibrosis in | | Generally performed at a |
| | provides a | the context of | | liver specialist clinic or |
| | measurement of | known and | | hospital. Obesity, liver |
| | liver stiffness that | underlying CLD. | | inflammation and ascites |
| | can correlate to | | | interfere with |
| | fibrosis. | | | measurements. |
| | | | | Variability in results limits |
| | | | | must be considered. |
| | | | | Expensive equipment limits |
| | | | | availability. |
| MRE | Combines MR | More accurate and | Combined with | Performed in a hospital or |
| (Magnetic | imaging with low- | complete views of | MR, MRE | specialized MRI facility. |
| resonance | frequency | the liver and | offers an | Typically ordered by a |
| elastography) | vibrations to | stiffness | overall | hepatology specialist. |
| | create a visual | measures. | assessment of | Data and test must be |
| | map that shows | | the liver and | interpreted by a radiologist. |
| | stiffness of body | | correlative | Extremely expensive |
| | tissues. | | assessment of | equipment limits availability. |
| | | | fibrosis. | |
| OnX Liver | Application of | Measures tissue | Inexpensive. | Currently for Investigational |
| Fibrosis | Machine Learning | acoustic properties | Available at | Use Only |
| | to raw RF signal | for a point-of-care | point-of-care. | |
| | from ultrasound | screening tool for | Test can be | |
| | combined with | the assessment of | completed by | |
| | demographic data | liver fibrosis in | any clinician | |
| | to provide a tissue | patients presenting | and done within | |
| | characterization | a risk of CLD. | the time span | |
| | score of liver | | of a single | |
| | fibrosis. | | clinical visit. | |
| | | | Noninvasive | |
| | | | and painless. | |

Several guidelines, KOLs and experts have begun calling for early screening for liver fibrosis of patients at primary care for signs of silent CLD. These experts are joined by patients and their advocacy groups around the globe, including The Fatty Liver Foundation, NASH kNOWledge and the Global Liver Institute, which was instrumental in bringing the NASH Care Act to the US Congress. Early detection, as noted above, facilitates early intervention, lifestyle modifications, treatments and referrals when needed. Early detection of CLD as noted by fibrosis stage can and does save patients lives. Anecdotal evidence is mounting via patient stories and advocacy groups that patients are interested in objective assessment of their liver health, as this information helps them make informed decisions about their health, lifestyle, behaviours and future plans. The OnX provides a much-needed point-of-care solution to detect an otherwise silent disease and empowers patients with additional insights into the state of their liver health.

The Liver Fibrosis Categorization System will provide patients and health care providers with a novel and innovative AI driven point-of-care tool that can provide effective and efficient screening of liver fibrosis in patients presenting a high risk of chronic liver disease. The system approach mines raw signals from off the shelf ultrasound sound devices and turns these into powerful tissue characterization tools for point-of-care clinicians. This new technology and approach addresses the growing epidemic of chronic liver disease—a ‘silent pandemic’ that has led KOLs to emphasize the need for tools such as the system. Thus, the early detection and categorization of this potentially life-threatening and debilitating condition promises to improve clinical efficiencies, decrease costs, and most importantly, to help patients access such assessments through point-of-care physicians and ultimately understand and manage their health, as well as improve the quality and length of their lives.

In some embodiments, the system allows for the collection of image raw data for an abdomen (not just a specific location of interest on the liver). The collection of images is filtered to select the best frames and a region of interest (ROI) is determined using the raw data to segment the liver from other parts of the abdomen. I.e., the system allows for easier determination of the ROI. The ROI may then be divided into windows or sections, and properties for those windows or sections are obtained. First and second order features are mapped for each window. These features are fed into a ML model for each window, each frame, each patient.

In some embodiments, the system allows for the collection of a relatively large quantity of image data, rather than requiring a high quality of an image. I.e., a probe may be pointed and shot following a protocol. This allows for time savings for a stenographer. For example, where the stenographer may typically require approximately 45 minutes for quality image taking, the protocol may obtain the quantity of images in approximately 5 minutes. Moreover, less experience is required for scanning (e.g., a stenographer may not be required).

In some embodiments, the system selects frames that maximize views (and ultimately data) of liver tissue. Frames may also be selected such that poor contact, shadowing and other artifacts in images may be removed.

In some embodiments, the selection is automated. For example, specific features may be defined such that a process may be configured to detect those features (e.g., shadow detection, ML trained to locate artifacts, etc.). In some embodiments, a measurement threshold may be set to filter (i.e., remove) poor frames.

In some embodiments, ROIs are labeled to train the ML model to select a ROI where liver tissue is in a frame. In some embodiments, the ROI masks as much liver as the ML model can recognize.

In some embodiments, the ROI is then divided into sections, patches or windows (e.g., into a grid). Acoustic properties for each window in the ROI are determined. The acoustic properties (i.e., features) can be determined per window, per frame or per patient. One the ML model is trained to classify on each level (e.g., window, frame or patient level), deep learning may be used to analyze the raw data and translate it to a patient level (e.g., diagnosis or score that matches a clinical standard).

In some embodiments, the ML may be used to classify the liver tissue as 1) healthy, 2) fibrotic or 3) cirrhotic. It should be understood that other classifications may be used. In some embodiments, regression may be used where the output is a number between two limits (e.g., a number between 1 and 10). For example, if the number is 1, then a first disease is noted; if the number is 2, then another disease may be noted; if the number is 0, then the tissue is healthy. In some embodiments, such regression may be used prior to classification.

In some embodiments, a regular score for classification may be expressed as a probability value. A regression score may be an attempt to predict the score.

In some embodiments, to classify a disease involves having a library of labelled tissue samples to train the model with known probabilities for each tissue sample. The classification score (e.g., OnX score) may then be used to classify a new tissue sample based on similarity assessment of the features. For example, a score of 0-3 may equate to a healthy tissue, a score of 3-7 may mean fibrotic, and a score of 7-10 may mean cirrhotic. Other classifications may be used.

In some embodiments, a closed loop system may be provided where the resulting classification may trigger a response or medical intervention. For example, an automated dosage of medication may be administered or ordered, a referral to/appointment with an medical expert may be automatically made, etc.

Combining Imaging with Diagnostic Methods

In some embodiments, ML may be applied to synthesize multiple imaging and diagnostic modalities in combination to enhance sensitivity and specificity of disease diagnostics.

Discrete imaging and diagnostic methods include blood tests and different imaging modalities each of which can provide biomarkers, including molecular, histologic, radiographic and physiologic. All such biomarkers are indicators of normal biological processes, pathogenic processes, or responses to an exposure or intervention, including therapeutic interventions. The method, process and algorithms proposed combine lower specificity and sensitivity AI based diagnostics on different imaging modalities (MRI/SWE/PoCUS/Ultrasound), other diagnostic techniques (bodily fluid analysis) and patient history, along with any assessments and/or resultant AI/ML derived scores, as potential inputs and a diagnostic assessment and an associated confidence value as an output.

In some embodiments, the method/process is designed to work both with sparse and with complete data. The confidence output will depend on the spasticity of the input information. The highest confidence value will be given to automated AI diagnostics based on a complete dataset including all the information modalities. Nonetheless, the method will give high confidence values on predictions that combine three of the data modalities named above and the lowest confidence level for those cases with only one of the data modalities as input.

To do the diagnostic assessment, the model will use predictions on each of the imaging modalities produced by other Machine Learning algorithms particularly trained to work on those. The overall input structure will contain predictions for each of the imaging modalities, concentration of substances in bodily fluids (blood, urine. etc.) and categorical and continuous demographic and clinical history data. There may be two outputs: 1. Diagnostic assessment, 2. Confidence level of the diagnostic.

It should be noted that only one of the imaging modalities will be necessary to be available for the algorithm to produce a prediction. Nevertheless, additional inputs will boost performance.

While one of the multi-modal approaches may be lacking in sensitivity or specificity, the goal here is to strategically combine these in a way that would ultimately enhance overall sensitivity and specificity of a screening or diagnostic test. Practically, this could allow for a rapid stratification of patients, and enhance diagnostic potential of complementary systems that are already established.

In some embodiments, AI may be trained to leverage multiple imaging and diagnostic modalities in combination to offer high sensitivity and specificity diagnostics.

This AI algorithm combines lower specificity and sensitivity AI based diagnostics on different imaging modalities (MRI/SWE/PoCUS/Ultrasound), other diagnostic techniques (bodily fluid analysis) and patient history as potential inputs and a diagnostic and an associated confidence value as an output.

In some embodiments, the algorithm is designed to work both with sparse and with complete data. The confidence output will depend on the spasticity of the input information. The highest confidence value will be given to automated AI diagnostics based on a complete dataset including all the information modalities. Nonetheless, the algorithm may adjust and potentially enhance confidence values on predictions that combine three of the data modalities named above compared to those cases with only one of the data modalities as input.

To do the diagnostics the model will use predictions on everyone of the imaging modalities produced by other Machine Learning algorithms particularly trained to work on those. The overall input structure will contain predictions for each of the imaging modalities, concentration of substances in bodily fluids (blood, urine. etc.) and categorical and continuous demographic and clinical history data. There will be two outputs: 1. Liver Disease diagnostic, 2. Confidence level of the diagnostic.

It should be noted that only one of the imaging modalities will be necessary to be available for the algorithm to produce a prediction. Nevertheless, any additional inputs will boost performance.

While one of the multi-modal approaches may be lacking in sensitivity or specificity, the goal here is to strategically combine these in a way that would ultimately enhance overall sensitivity and specificity of a screening or diagnostic test. Practically, this could allow for the rapid stratification of patients, and enhance diagnostic potential of complementary systems that are already established.

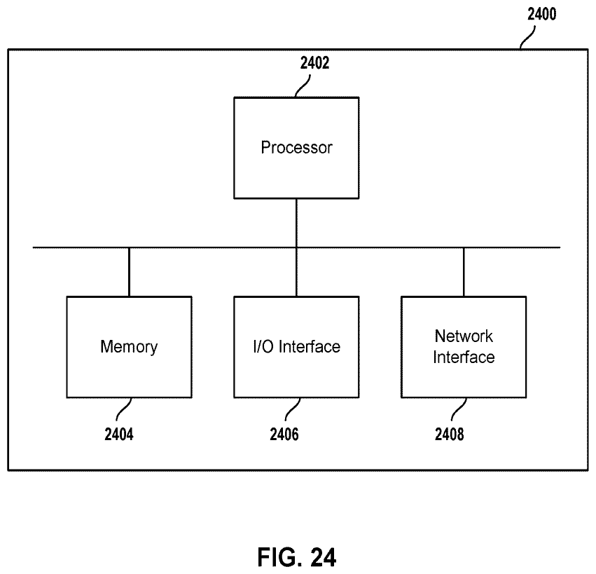

FIG. 24 is a schematic diagram of a computing device 2400 such as a server or other computer in a device such as a vehicle. As depicted, the computing device includes at least one processor 2402, memory 2404, at least one I/O interface 2406, and at least one network interface 2408.

Processor 2402 may be an Intel or AMD x86 or x64, PowerPC, ARM processor, or the like. Memory 2404 may include a suitable combination of computer memory that is located either internally or externally such as, for example, random-access memory (RAM), read-only memory (ROM), compact disc read-only memory (CDROM).

Each I/O interface 2406 enables computing device 2400 to interconnect with one or more input devices, such as a keyboard, mouse, camera, touch screen and a microphone, or with one or more output devices such as a display screen and a speaker.

Each network interface 2408 enables computing device 2400 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signalling network, fixed line, local area network, wide area network, and others.

The foregoing discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

In accordance with some embodiments, there is provided an ultrasound guidance system for organ-specific tissue data capture.

In accordance with some embodiments, there is provided a system for providing real time feedback on raw data signal detection and usability while or near-simultaneous to tissue data capture.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

Any and all features of novelty or inventive step described, suggested, referred to, exemplified, or shown herein, including but not limited to processes, systems, devices, and computer-readable and -executable programming and/or other instruction sets suitable for use in implementing such features.

As can be understood, the examples described above and illustrated are intended to be exemplary only.

What is claimed is:

1. A system for characterising tissues, the system comprising:
a point-of-care ultrasound device for obtaining at least one of ultrasound images and/or raw data of tissues;
a processor; and
a memory comprising instructions which when executed by the processor configure the processor to:
obtain an ultrasound image of a tissue of interest;
identify features of interest on the ultrasound image;
feeding said identified features into a trained machine learning (ML) model; and
identify a tissue pathology based on the identified features fed through the model.

2. The system of claim 1, further comprising adding additional clinical data sets and/or biomarkers.

3. The system as claimed in claim 1, wherein the ultrasound device is configured to provide data capture guidance.

4. The system as claimed in claim 1, wherein the features of interest are based on trained features fed into ML system.

5. The system as claimed in claim 1, wherein the processor is configured to:
obtain a plurality of ultrasound images of tissues, each ultrasound image labelled with at least one tissue pathology from a plurality of tissue pathologies; and
train a model based on the labelled ultrasound images.

6. The system as claimed in claim 5, wherein training said model is based on the labelled ultrasound images without additional clinical datasets and/or biomarkers.

7. The system as claimed in claim 5, wherein training said model is based on the labelled ultrasound images and on additional clinical datasets and/or biomarkers.

8. The system as claimed in claim 3, wherein the processor is configured to:
determine a quality of each frame of the plurality of ultrasound images; and
discard any frame below a quality threshold prior to training the model.

9. The system as claimed in claim 1, wherein the processor is configured to:
determine a proximity score between the identified features and features in the trained model, wherein the tissue pathology is identified based on the proximity score.

10. The system as claimed in claim 1, wherein the pathology is one of: normal, fibrosis, steatosis, inflammation, cancer, adenomas, or cirrhosis, and the proximity score is a corresponding one of: a normal score, a fibrosis score, a steatosis score, an inflammation score, a cancer score, an adenoma score, or a cirrhosis score.

11. The system as claimed in claim 1, wherein physical measures and/or measurements of the ultrasound coefficient of attenuation are presented as one or more of a range of scores, an estimate, and/or a direct measurement.

12. The system as claimed in claim 11, wherein the physical measures include estimates of tissue stiffness in kiloPascals (kPa).

13. The system as claimed in any one of claims 1 to 12, wherein the tissue is one of several types found in: a liver, a thyroid, a breast, a kidney, a prostate, a bowel, a pancreas, an ovary, a musculoskeletal, skin and wounds, or other organs or glands.

14. A computer-implemented method of characterising liver tissues, the method comprising:
obtaining an ultrasound image of a system of interest and tissue from within that system;
identifying features of the said system of interest and tissue on the ultrasound image;
feeding said identified features to a trained model; and
identifying a tissue pathology based on the identified features.

15. The computer-implemented method of claim 14, further comprising adding additional clinical datasets and biomarkers

16. The computer-implemented method as claimed in claim 14, wherein the ultrasound device is configured to provide data capture guidance.

17. The computer-implemented method as claimed in claim 14, wherein the features of interest are based on trained features fed into ML system.

18. The computer-implemented method as claimed in claim 14, comprising:
obtaining a plurality of ultrasound images of systems of interest and tissues, each ultrasound image labelled with at least one tissue pathology from a plurality of tissue pathologies; and
training a model based on the labelled ultrasound images.

19. The system of claim 18, wherein training said model is based on said labelled ultrasound images without additional clinical datasets and/or biomarkers.

20. The system of claim 18, wherein training said model is based on said labelled ultrasound images and additional clinical datasets and/or biomarkers.

21. The computer-implemented method as claimed in claim 17, comprising:
determining a quality of each frame of the plurality of ultrasound images; and
discarding any frame below a quality threshold prior to training the model.

22. The system of claim 21, further comprising directing a user to retake one or more ultrasound images when said frame is below the quality threshold.

23. The system of claim 14, further comprising directing a user to retake all of said ultrasound images when the system determines that insufficient data was captured.

24. The computer-implemented method as claimed in claim 14, comprising:
determining a proximity score between the identified features and features in the trained model, wherein the tissue pathology is identified based on the proximity score.

25. The computer-implemented method as claimed in claim 14, comprising: determining an estimate of one or more of tissue stiffness and/or an ultrasound coefficient of attenuation; and presenting said tissue stiffness and/or ultrasound coefficient of attenuation as one or more of a range of scores, an estimate, and/or a direct measurement.

26. The computer-implemented method as claimed in claim 25, wherein the tissue stiffness is represented in kiloPascals (kPA).

27. The computer-implemented method as claimed in claim 14, wherein estimates of tissue stiffness and/or measurements of an ultrasound coefficient of attenuation are presented as one or more of a range of scores, an estimate, or a direct measurement.

28. The computer-implemented method as claimed in claim 14, wherein the pathology is one of: normal, fibrosis, steatosis, inflammation, cancer, adenomas, or cirrhosis, and the proximity score is a corresponding one of: a normal score, a fibrosis score, a steatosis score, an inflammation score, a cancer score, an adenomas score or a cirrhosis score.

29. The system as claimed in any one of claims 14 to 28, wherein tissue is one of several types found in: a liver, a thyroid, a breast, a kidney, a prostate, a bowel, a pancreas, an ovary, a musculoskeletal, skin and wounds, or other organs or glands.
